(19) United States
(12) Patent Application Publication
(10) Pub. No.: US 2026/0204359 A1
(43) Pub. Date: Jul. 16, 2026

(54) ODOR IDENTIFICATION METHOD AND ODOR IDENTIFICATION SYSTEM

(71) Applicant: Panasonic Intellectual Property Management Co., Ltd., Osaka (JP)

(72) Inventor: Toshiki NIINOMI, Miyagi (JP)

(21) Appl. No.: 19/135,039

(22) PCT Filed: Nov. 1, 2023
(86) PCT No.: PCT/JP2023/039456

§ 371 Date: Jun. 2, 2025

(30) Foreign Application Priority Data
Dec. 26, 2022 (JP) 2022-208160

(51) Int. Cl. G16C 20/20; G01N 33/00; G16C 20/70

(52) U.S. Cl. CPC G16C 20/20; G01N 33/0034; G16C 20/70

(57) ABSTRACT

An odor identification method includes: obtaining a plurality of detection signals output from a plurality of sensors exposed to a sample gas; determining, for each of the plurality of sensors, whether the sensor is anomalous based on a feature extracted from the signal output from the sensor; selecting one of a plurality of trained logical models that are for identifying an odorant and different from each other, based on a result of determination in the determining; and identifying the odorant contained in the sample gas, based on the plurality of detection signals obtained in the obtaining of the plurality of detection signals and the one of the plurality of trained logical models selected in the selecting.

### TECHNICAL FIELD

The present disclosure relates to an odor identification method and an odor identification system.

### BACKGROUND ART

An odor identification method is known which identifies an odorant contained in a sample gas, based on a signal output from a sensor exposed to the sample gas. For example, Patent Literature (PTL) 1 discloses identifying the source of the odor, based on the patterns of the respective signals output from a plurality of sensors.

### CITATION LIST

### Patent Literature

[PTL 1] Japanese Unexamined Patent Application Publication No. 2020-012846

### SUMMARY OF INVENTION

### Technical Problems

In the identification of the odorant, identification accuracy improves when using a plurality of sensors than in the case of using one sensor. Even in the use of a plurality of sensors, however, identification accuracy may deteriorate due to various causes, such as the conditions of the sensors or the environment.

To address the problem, the present disclosure provides an odor identification method and an odor identification system that can reduce a deterioration in the accuracy of identifying an odorant.

### Solution to Problems

An odor identification method according to an aspect of the present disclosure is a method of identifying an odorant contained in a sample gas, using a plurality of sensors each outputting a signal according to an adsorption concentration of molecules. The odor identification method includes: obtaining a plurality of detection signals output from the plurality of sensors exposed to the sample gas; determining, for each of the plurality of sensors, whether the sensor is anomalous based on a feature extracted from the signal output from the sensor; selecting one of a plurality of trained logical models that are for identifying the odorant and different from each other, based on a result of determination in the determining; and identifying the odorant contained in the sample gas, based on the plurality of detection signals obtained in the obtaining of the plurality of detection signals and the one of the plurality of trained logical models selected in the selecting.

An odor identification system according to an aspect of the present disclosure is for identifying an odorant contained in a sample gas, using a plurality of sensors each outputting a signal according to an adsorption concentration of molecules. The odor identification system includes: an obtainer that obtains a plurality of detection signals output from the plurality of sensors exposed to the sample gas; a determiner that determines, for each of the plurality of sensors, whether the sensor is anomalous based on a feature extracted from the signal output from the sensor; a selector that selects one of a plurality of trained logical models that are for identifying the odorant and different from each other, based on a result of determination by the determiner; and an identifier that identifies the odorant contained in the sample gas, based on the plurality of detection signals obtained by the obtainer, and the one of the plurality of trained logical models selected by the selector.

Note that these general and specific aspects of the present disclosure may be implemented using a system, a device, a method, an integrated circuit, a computer program, or a non-transitory computer-readable recording medium, such as a compact disc read-only memory (CD-ROM), or any combination of systems, devices, methods, integrated circuits, computer programs, or recording media.

### Advantageous Effects of Invention

The present disclosure can reduce a deterioration in the accuracy of identifying an odorant.

### BRIEF DESCRIPTION OF DRAWINGS

FIG. 1 is a block diagram showing a schematic configuration of an odor identification system according to the embodiment.

FIG. 2 is a top view showing an example sensor according to the embodiment.

FIG. 3 is a schematic view showing an example configuration of an exposer according to the embodiment.

FIG. 4 is a flowchart showing an example operation of the odor identification system according to the embodiment.

FIG. 5 shows an example detection signal output from a sensor according to the embodiment.

FIG. 6 shows example determination data used for determination by a determiner according to the embodiment.

FIG. 7 shows example selection data used for selection by the selector according to the embodiment.

FIG. 8 is for illustrating an outline of selection processing by the selector according to the embodiment.

FIG. 9 is a flowchart for illustrating another example operation of the odor identification system according to the embodiment.

FIG. 10 shows rates of change of the plurality of detection signals for an identification test.

FIG. 11 is for illustrating how to conduct the identification test of an odorant.

FIG. 12 shows a result of identifying an odorant in the identification test.

### DESCRIPTION OF EMBODIMENT

### (Circumstances Leading to the Present Disclosure)

Circumstances leading to an aspect of the present disclosure will be described prior to specific description of an embodiment of the present disclosure. The present inventor found the following problem when identifying an odorant using a plurality of sensors.

With respect to an odor identification method, assume that a plurality of sensors with different sensitivities to an odorant are used. In this case, even when the sensors are exposed to the sample gas, the signals output from the plurality of sensors are different from each other. This increases the features for identification. Identification of the odorant based on the features improves the identification accuracy. However, when a plurality of sensors are used, which include an anomalous sensor due to a malfunction, a deterioration, a damage, or excessive or insufficient interaction between the sensor and the odorant, the identification accuracy may deteriorate against the purpose. When a plurality of sensors are used for the odorant identification, it is thus not always the best to use all the sensors for the odorant identification.

The present disclosure was made based on such findings. It is an objective to provide an odor identification method and an odor identification system that can reduce the deterioration in the accuracy in identifying the odorant, even when the plurality of sensors include an anomalous sensor.

### [Outline of Present Disclosure]

Now, as an outline of the present disclosure, an example of the odor identification method and the odor identification system according to the present disclosure will be given.

An odor identification method according to an aspect of the present disclosure is a method of identifying an odorant contained in a sample gas, using a plurality of sensors each outputting a signal according to an adsorption concentration of molecules. The odor identification method includes: obtaining a plurality of detection signals output from the plurality of sensors exposed to the sample gas; determining, for each of the plurality of sensors, whether the sensor is anomalous based on a feature extracted from the signal output from the sensor; selecting one of a plurality of trained logical models that are for identifying the odorant and different from each other, based on a result of determination in the determining; and identifying the odorant contained in the sample gas, based on the plurality of detection signals obtained in the obtaining of the plurality of detection signals and the one of the plurality of trained logical models selected in the selecting.

Accordingly, the one trained logical model used for identifying the odorant contained in the sample gas is selected based on a result of determining whether each of the plurality of sensors is anomalous. The trained logical model used for the odorant identification may be different between the case where all the plurality of sensors are normal and the case where the plurality of sensors include a sensor outputting an anomalous signal. Accordingly, in this aspect, even when the plurality of sensors include an anomalous sensor, deterioration in the accuracy in identifying the odorant can be reduced by selecting the trained logical model associated with the sensor determined to be anomalous. For example, even if any of the plurality of sensors becomes anomalous like malfunctioning, deteriorating, or damaged due to long-term use, the accuracy in identifying the odorant less deteriorates.

For example, an odor identification method according to a second aspect of the present disclosure is an embodiment of the odor identification method according to the first aspect. In the determining, a feature extracted from each of the plurality of detection signals is used as the feature.

Accordingly, whether the sensor is anomalous can be determined using the feature extracted from the detection signal also used for the odorant identification, which can simplify the odor identification method.

For example, an odor identification method according to a third aspect of the present disclosure is an embodiment of the odor identification method according to the first aspect. The odor identification method further includes: obtaining a plurality of determination signals output from the plurality of sensors exposed to a determination gas with a predetermined humidity. In the determining, a feature extracted from each of the plurality of determination signals is used as the feature.

Accordingly, since the anomaly of the sensor is determined using highly responsive water molecules in the sensor, the anomaly of the sensor can be determined with high accuracy.

For example, an odor identification method according to a fourth aspect of the present disclosure is an embodiment of the odor identification method according to any one of the first to third aspects. The determining includes determining, for each of the plurality of sensors, whether the sensor is anomalous, based on whether the feature associated with the sensor meets a predetermined requirement. A predetermined requirement associated with a first sensor of the plurality of sensors is different from a predetermined requirement associated with a second sensor of the plurality of sensors.

Accordingly, whether the sensor is anomalous can be determined using the determination criteria according to the sensitivity characteristics of the sensor, which increases the determination accuracy.

For example, an odor identification method according to a fifth aspect of the present disclosure is an embodiment of the odor identification method according to any one of the first to fourth aspects. The selecting includes selecting, as the one of the plurality of trained logical models, a trained logical model containing no input associated with a sensor determined to be anomalous in the determining.

Accordingly, the odorant can be identified using a trained logical model containing no input associated with a sensor determined to be anomalous, which further increases the accuracy in identifying the odorant.

For example, an odor identification method according to a sixth aspect of the present disclosure is an embodiment of the odor identification method according to any one of the first to fourth aspects. The selecting includes selecting, as the one of the plurality of trained logical models, a trained logical model in which an input associated with a sensor determined to be anomalous in the determining is weighted with a predetermined threshold or less.

Accordingly, the odorant can be identified using a trained logical model in which the input associated with a sensor determined to be anomalous is less weighted, which further increases the accuracy in identifying the odorant.

For example, an odor identification method according to a seventh aspect of the present disclosure is an embodiment of the odor identification method according to any one of the first to sixth aspects. In the determining, the feature includes an amount of change, a rate of change, or a slope of the signal output from the sensor.

Accordingly, whether the sensor is anomalous can be determined with high accuracy.

For example, an odor identification method according to an eighth aspect of the present disclosure is an embodiment of the odor identification method according to any one of the first to seventh aspects. The obtaining of the plurality of detection signals includes obtaining a plurality of detection signals output from the plurality of sensors via a network.

Accordingly, the plurality of detection signals can be obtained easily, even if there is a sensor in a remote place.

An odor identification system according to a ninth aspect of the present disclosure is for identifying an odorant contained in a sample gas, using a plurality of sensors each outputting a signal according to an adsorption concentration of molecules. The odor identification system includes: an obtainer that obtains a plurality of detection signals output from the plurality of sensors exposed to the sample gas; a determiner that determines, for each of the plurality of sensors, whether the sensor is anomalous based on a feature extracted from the signal output from the sensor; a selector that selects one of a plurality of trained logical models that are for identifying the odorant and different from each other, based on a result of determination by the determiner; and an identifier that identifies the odorant contained in the sample gas, based on the plurality of detection signals obtained by the obtainer, and the one of the plurality of trained logical models selected by the selector.

Accordingly, the odor identification system can reduce the deterioration in the accuracy in identifying the odorant, like the odor identification method according to the first aspect described above.

Now, an embodiment will be described in detail with reference to the drawings. Note that the embodiment described below is a mere general or specific example of the present disclosure. The numerical values, shapes, materials, elements, the arrangement and connection of the elements, steps, step orders etc. shown in the following embodiments are thus mere examples, and are not intended to limit the scope of the present disclosure. Among the elements in the following embodiment, those not recited in the independent claims will be described as optional.

In this specification, the terms, such as “parallel” representing the relationship between the elements, the terms representing the shapes of the elements, and the numerical ranges do not have strict meaning but represent substantially equivalent ranges or errors of several percentages.

The figures are not necessarily drawn strictly to scale. The same reference signs represent substantially the same configurations in the drawings and redundant description will be omitted or simplified.

In this specification, unless otherwise noted, the ordinal numbers, such as “first” or “second”, do not mean the number or order of the elements, but are for distinguishing the elements, while avoiding confusions of the elements of the same type.

### EMBODIMENT

### [Configuration]

First, a configuration of an odor identification system according to an embodiment will be described.

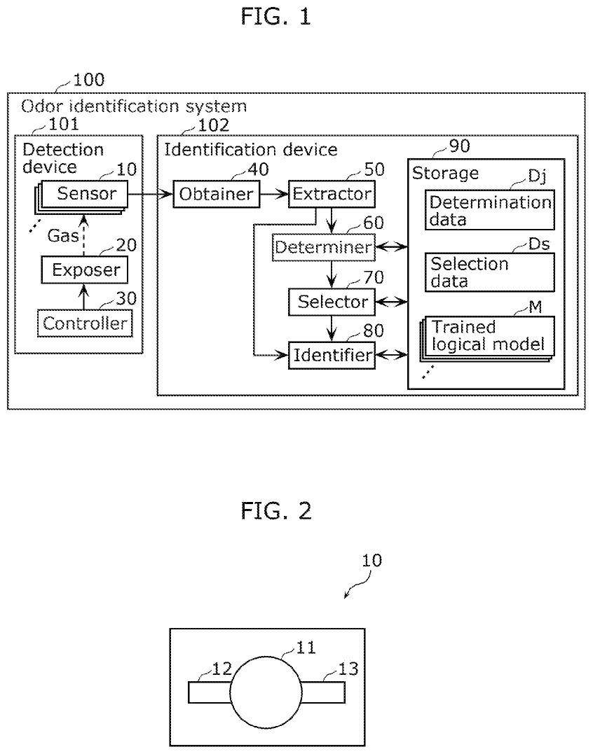

FIG. 1 is a block diagram showing a schematic configuration of odor identification system 100 according to this embodiment.

As shown in FIG. 1, odor identification system 100 according to this embodiment includes detection device 101, and identification device 102. Detection device 101 includes a plurality of sensors 10, exposer 20, and controller 30. Identification device 102 includes obtainer 40, extractor 50, determiner 60, selector 70, identifier 80, and storage 90.

Odor identification system 100 is for identifying an odorant contained in a sample gas. Odor identification system 100 identifies which kind odorant is contained in the sample gas, based on outputs of the plurality of sensors 10 exposed to a sample gas, for example.

The sample gas may contain an odorant, such as a volatile organic compound. The sample gas is, for example, collected from or around food, human breath, the air around a human body, or the air collected from a room in a building. Note that the odorant may be an inorganic compound, such as ammonia or hydrogen sulfide.

Each of the plurality of sensors 10 outputs a signal associated adsorption of the molecules of sensor 10. Specifically, the signals output from sensors 10 differ in accordance with the adsorption concentrations of the molecules. If the molecules to be adsorbed by sensors 10 are of different types, even sensors 10 with the same adsorption concentration output different signals. For example, sensors 10 are of an electrochemical type, a semiconductor type, a field-effect transistor type, a surface acoustic wave type, a quartz crystal type, or a resistance change type.

FIG. 2 is a top view showing an example of each sensor 10 according to this embodiment. As shown in FIG. 2, sensor 10 includes, for example, sensing section 11 and a pair of electrodes 12 and 13 electrically connected to sensing section 11. Sensing section 11 is, for example, in the shape of a sensing film whose electrical resistance changes in accordance with the adsorption concentration of molecules. The signal according to the electrical resistance of sensing section 11 of sensor 10 is obtained, for example, as a voltage signal or a current signal by obtainer 40 via the pair of electrodes 12 and 13. The electrical resistance of sensing section 11 between the pair of electrodes 12 and 13 is, for example, converted to a voltage signal or a current signal by a detector (not shown).

Sensing section 11 is, for example, made of a resin material and conductive particles. The resin material is an adsorbent that adsorbs the molecules to be identified by odor identification system 100. The conductive particles are dispersed in the resin material. Examples of the resin material include a polyalkylene glycol resin, a polyester resin, and a silicone resin. For example, the resin material has a side chain that is commercially available as a stationary phase for gas chromatography columns. In view of the durability and adsorption of the molecules, the resin material is, for example, a silicone resin that is commercially available as a stationary phase for columns, and has various substituents, such as a phenyl or methyl group, as a side chain. Alternatively, sensing section 11 is not necessarily made of a resin material and conductive particles, but may be a member whose electrical resistance changes due to the adsorption of the molecules to be identified. Sensing section 11 may be, for example, made of an inorganic material, such as a metal oxide, or porous ceramics.

At least two of the plurality of sensors 10 have sensitivity characteristics different from each other. Sensing sections 11 (specifically, the resin materials of sensing sections 11) of the at least two of the plurality of sensors 10 are made of, for example, different types of materials. In the case of resin materials, the “different types of materials” have, for example, at least substantially different molecule quantities or composition formulae. The “different types of materials” exhibit different adsorption behaviors to the same kind of molecules. That is, at least two of the plurality of sensors 10 exhibit different molecule adsorption behaviors. In particular, the materials with different composition formulae exhibit significantly different molecule adsorption behaviors. Sensing sections 11 of all the plurality of sensors 10 may be made of different types of materials. In this case, the plurality of sensors 10 output different signals even when adsorbing the same kind of molecules. Accordingly, the different features are extracted from the outputs of the plurality of sensors 10, which increases the identification accuracy of odor identification system 100.

Referring back to FIG. 1, exposer 20 is an exposure mechanism that exposes the plurality of sensors 10 to a gas in a predetermined measurement period based on the control by controller 30. For example, exposer 20 exposes the plurality of sensors 10 to a gas in a predetermined measurement period including a first period, a second period subsequent to the first period, and a third period subsequent to the second period. For example, exposer 20 exposes the plurality of sensors 10 to a sample gas containing an odorant in the second period of the measurement period. For example, exposer 20 exposes the plurality of sensors 10 to the sample gas only in the second period of the measurement period, and does not expose the plurality of sensors 10 to the sample gas in the first period or the third period. Accordingly, the concentration of molecules to be identified around the plurality of sensors 10 become higher in the second period than in the first period and the third period. The molecules to be identified are more likely to be adsorbed by the plurality of sensors 10 in the second period. That is, exposer 20 exposes the plurality of sensors 10 to the sample gas under the following conditions. The odorant contained in the sample gas is more likely to be adsorbed by the plurality of sensors 10 in the second period than in the first period and the third period, out of the measurement period of exposing the plurality of sensors 10 to the sample gas.

Exposer 20 may expose the plurality of sensors 10 to a reference gas in the first period and the third period. The reference gas has a different composition from the sample gas and serves as a reference of measurement. The reference gas contains, for example, no molecules to be identified or the molecules to be identified at a concentration significantly lower than in the sample gas (e.g., one tenth or less of the concentration of molecules in the sample gas). The composition of the reference gas does not substantially change in each measurement. The reference gas is, for example, made of molecules less likely to be adsorbed by sensing sections 11 of the plurality of sensors 10 than the molecules to be identified.

Specific examples of the reference gas include industrial or analytical air, and an inert gas, such as nitrogen or a noble gas, containing substantially no water molecules or an organic compound, and a gas obtained by removing the molecules to be identified from the sample gas using a filter, for example. In this manner, the plurality of sensors 10 are exposed to the reference gas in the first period and the third period and the surrounding environment changes, for example. Even in this case, the signals output from the plurality of sensors 10 become stable in each measurement and the identification accuracy increases.

An example will be mainly described below in this embodiment where exposer 20 exposes the plurality of sensors 10 to the sample gas in the second period and to the reference gas in the first period and the third period.

Controller 30 controls the operation of exposer 20. Controller 30 is a microcomputer or a processor, for example, including built-in programs for the processing described above or later. Controller 30 may be a dedicated logic circuit for the processing described above or later.

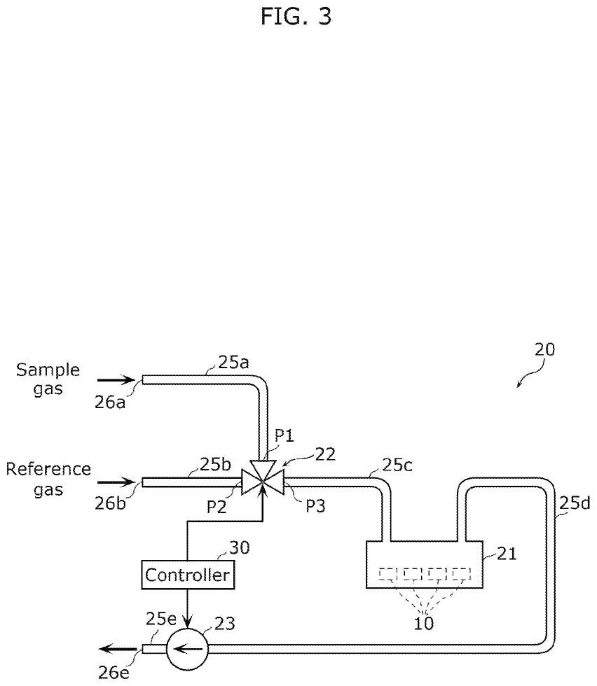

Here, a specific configuration of exposer 20 will be described. FIG. 3 is a schematic view showing an example configuration of exposer 20 according to this embodiment. As shown in FIG. 3, exposer 20 includes, for example, housing 21, three-way electromagnetic valve 22, suction pump 23, and a plurality of pipes 25a, 25b, 25c, 25d, and 25e.

Pipe 25a has, at one end, suction port 26a for introducing the sample gas. Suction port 26a is provided, for example, in the space filled with the sample gas. Pipe 25b has, at one end, suction port 26b for introducing the reference gas. Suction port 26b is provided, for example, in the space filled with the reference gas. Pipe 25e has, at one end, exhaust port 26e for discharging the introduced sample gas and reference gas.

Housing 21 is a box-shaped container containing the plurality of sensors 10. Inside housing 21, for example, a plurality of sensors 10 are arranged in an array. One ends of pipe 25c and pipe 25d are connected to housing 21. Once suction pump 23 operates, which will be described, the gas flows from one end of pipe 25c to one end of pipe 25d. The plurality of sensors 10 are arranged in the flow path of the gas.

The sample gas introduced from suction port 26a is then introduced via pipe 25a, three-way electromagnetic valve 22, and pipe 25c into housing 21. The reference gas introduced from suction port 26b is then introduced via pipe 25b, three-way electromagnetic valve 22, and pipe 25c into housing 21. The sample gas and reference gas introduced into housing 21 are discharged from exhaust port 26e via pipe 25d, suction pump 23, and pipe 25e.

Three-way electromagnetic valve 22 is for switching the gas to be introduced into housing 21. Three-way electromagnetic valve 22 includes inlet port P1 connected to the other end of pipe 25a, inlet port P2 connected to the other end of pipe 25b, and outlet port P3 connected to the other end of pipe 25c. The opening and closing of the ports of three-way electromagnetic valve 22 are based on the control by controller 30. Three-way electromagnetic valve 22 switches between a first state and a second state based on the control by controller 30. In the first state, inlet port P1 and outlet port P3 are conductive. In the second state, inlet port P2 and outlet port P3 are conductive. In the first state, inlet port P1 and outlet port P3 are open, and inlet port P2 is closed. On the other hand, in the second state, inlet port P2 and outlet port P3 are open, and inlet port P1 is closed.

Suction pump 23 is for introducing the sample gas and reference gas into housing 21, and discharging the introduced sample gas and reference gas from exhaust port 26e. The operation of suction pump 23 is based on the control by controller 30. The suction port of suction pump 23 is connected to the other end of pipe 25d. On the other hand, the exhaust port of suction pump 23 is connected to the other end of pipe 25e.

With such a configuration, when three-way electromagnetic valve 22 is in the first state, while suction pump 23 is operating, the sample gas is introduced into housing 21. Accordingly, exposer 20 exposes the plurality of sensors 10 to the sample gas. When three-way electromagnetic valve 22 is in the second state, while suction pump 23 is operating, the reference gas is introduced into housing 21. Accordingly, exposer 20 exposes the plurality of sensors 10 to the reference gas. By controlling such three-way electromagnetic valve 22, the plurality of sensors 10 are exposed only to the sample gas when three-way electromagnetic valve 22 is in the first state, and only to the reference gas when three-way electromagnetic valve 22 is in the second state.

Note that the configuration of exposer 20 is not particularly limited to that shown in FIG. 3, as long as being capable of exposing the plurality of sensors 10 to the sample gas. In exposer 20, for example, the sample gas and the reference gas may be introduced into housing 21 not via three-way electromagnetic valve 22 but via another pipe. Exposer 20 not necessarily includes suction pump 23 but may always allow a carrier gas to flow into housing 21 and mix the sample gas into the carrier gas. There is also no need to introduce the reference gas. After the plurality of sensors 10 are exposed to the sample gas, suction pump 23 may evacuate housing 21. In addition, exposer 20 may include a temperature controller that controls the temperature of housing 21, expose the plurality of sensors 10 to the sample gas in the entire measurement period. By controlling the temperature of housing 21 in the second period to be lower than in the first period and the third period, the odorant contained in the sample gas is more likely to be adsorbed by sensors 10 in the second period than in the first period and the third period. Exposer 20 may further include various removal filters for removing the moisture or fine particles in the sample gas and the reference gas, electromagnetic control valves for adjusting the flow rates of the pipes, and check valves against the backflow of the pipes. This exposer 20 may also have a mechanism for adjusting the humidity inside housing 21.

Referring back to FIG. 1, obtainer 40 obtains the respective signals output from the plurality of sensors 10. Obtainer 40 obtains, for example, voltage signals or current signals, as the signals output in association with the electrical resistances of respective sensing sections 11 of the plurality of sensors 10. Obtainer 40 obtains, for example, a plurality of detection signals output from the plurality of sensors 10 exposed to a sample gas. The plurality of detection signals are associated with sensors 10 on a one-to-one basis.

Extractor 50 extracts a feature of the signal output from each of the plurality of sensors 10 obtained by obtainer 40. Extractor 50 extracts one or more features from the signal associated with one sensor 10. The signal indicating the feature(s) extracted by extractor 50 is, for example, the detection signal described above or a determination signal which will be described later.

Determiner 60 determines, for each of the plurality of sensors 10, whether this sensor 10 is anomalous, for example, based on a feature associated with this sensor 10 and extracted by extractor 50. Determiner 60 determines, for example, whether the feature associated with this sensor 10 meets a predetermined requirement. That is, for example, the feature of anomalous sensor 10 fails to meet the predetermined requirement. In this specification, the anomaly of sensor 10 include not only a malfunction, a deterioration, or a damage of sensor 10 but also include the cases in which sensor 10 is hardly sensitive to an odorant to be identified or output of sensor 10 is saturated since sensor 10 is too sensitive to an odorant to be identified.

Selector 70 selects one of a plurality of different trained logical models M stored in storage 90, based on a result of determination by determiner 60 as to whether each of the plurality of sensors is anomalous. Each of the plurality of trained logical models M is for identifying an odorant.

Identifier 80 identifies the odorant contained in the sample gas, based on features extracted from the plurality of detection signals obtained by obtainer 40 and trained logical model M selected by selector 70. Identifier 80 identifies which kind of odorant is contained in the sample gas, for example, by inputting the features extracted from the plurality of detection signals by extractor 50 into trained logical model M selected by selector 70. Identifier 80 outputs the information indicating a result of identification to a display (not shown), such as a display screen, for example, in odor identification system 100. Accordingly, the display displays a result of identification by identifier 80. Identifier 80 may cause storage 90 to store the information indicating a result of identification. Alternatively, identifier 80 may output the information indicating a result of identification to an external device.

Obtainer 40, extractor 50, determiner 60, selector 70, and identifier 80 may be each a microcomputer or a processor, for example, including built-in programs for processing described above or later. Each of obtainer 40, extractor 50, determiner 60, selector 70, and identifier 80 may be a dedicated logic circuit for the processing described above or later.

Storage 90 is a storage device storing information and data necessary for the processing performed by identification device 102. Storage 90 is a semiconductor memory or a hard disk drive (HDD), for example.

Storage 90 stores a plurality of trained logical models M for use in odorant identification. Specifically, each trained logical model M is for identifying which kind of odorant is contained in a sample gas, for example. For example, each trained logical model M receives, as inputs, features of the plurality of detection signals obtained by obtainer 40 and outputs the information indicating which kind of odorant is contained in the sample gas. Each trained logical model M may receive, as inputs, features of the plurality of detection signals obtained by obtainer 40 and output the information whether the sample gas contains an odorant to be identified.

Each trained logical model M is built, for example, by machine learning on a logical model using a known odorant and the following features as training data. The features are of the respective detection signals output from the plurality of sensors 10 exposed to the sample gas containing the known odorant. For example, a neural network, a random forest, a support vector machine, a self-organizing map, or any other suitable means is used for the logical model subjected to the machine learning.

The plurality of trained logical models M are different from each other. For example, the plurality of trained logical models M receive, as inputs, the plurality of features associated with different sensors 10. Examples of the plurality of trained logical models M include the following. Trained logical models M may be subjected to machine learning using, as training data, the features extracted from the detection signals of all the plurality of sensors 10 and receive the features as inputs. Trained logical models M may be subjected to machine learning using, as training data, the features extracted from the detection signals of some of the plurality of sensors 10 and receive the features as inputs.

The plurality of trained logical models M may assign, for example, the inputs associated with the plurality of sensors 10 are weighted differently. The weights of the inputs associated with the plurality of sensors 10 may be checked, for example, based on the coefficients of the logical expressions of trained logical models M. Alternatively, the weights to the inputs of the plurality of sensors 10 may be checked, for example, by changing the values input to trained logical models M.

The plurality of trained logical models M are generated, in which the respective inputs associated with the plurality of sensors 10 are weighed differently, for example, by changing a coefficient of an input of predetermined sensor 10 in trained logical model M subjected to machine learning using, as the training data, the features extracted from the detection signals of all the plurality of sensors 10.

Storage 90 may store determination data Dj to be used for determination by determiner 60, and selection data Ds to be used for selection by selector 70.

### [Operation]

Now, an operation (i.e., processing) of odor identification system 100 according to this embodiment, that is, an odor identification method will be described.

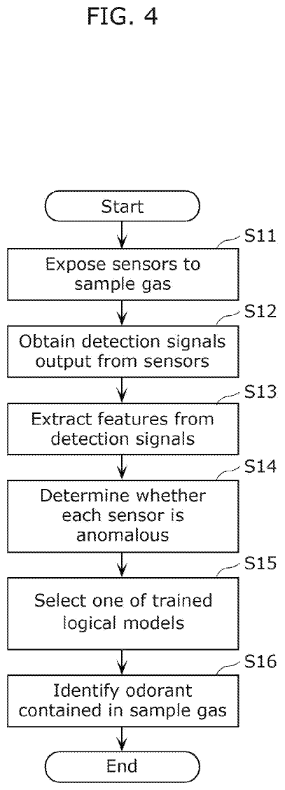

FIG. 4 is a flowchart showing an example operation of odor identification system 100 according to this embodiment. In the following description, step S12 is an example of “obtaining the plurality of detection signals”. Step S14 is an example of “determining”. Step S15 is an example of “selecting”. Step S16 is an example of “identifying”.

As shown in FIG. 4, first, exposer 20 exposes the plurality of sensors 10 to the sample gas based on the control by controller 30 (step S11). For example, exposer 20 exposes the plurality of sensors 10 to the sample gas under the following conditions. The conditions, the molecules are more likely to be adsorbed by sensors 10 in the second period than in the first period and the third period of a measurement period of exposing the plurality of sensors 10 to the sample gas. Exposer 20 also exposes sensors 10 to a reference gas in the first period and the third period. For example, controller 30 operates suction pump 23 to control the opening and closing of the ports of three-way electromagnetic valve 22. Accordingly, the plurality of sensors 10 are exposed to the reference gas in the first period and the third period, and to the sample gas in the second period.

Next, obtainer 40 obtain a plurality of detection signals output from the plurality of sensors 10 exposed to the sample gas in step S11 (step S12).

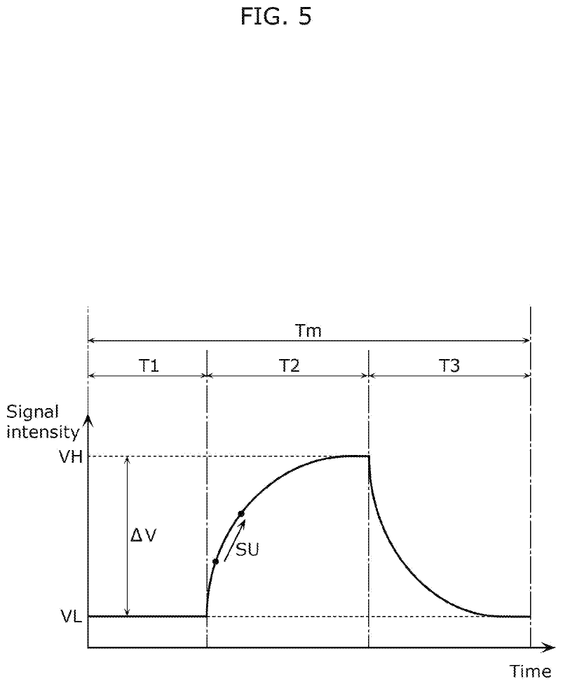

FIG. 5 shows an example detection signal output from sensor 10. FIG. 5 shows an example time change in the intensity (e.g., the voltage) of the detection signal output from one of the plurality of sensors 10 in measurement period Tm including first period T1, second period T2, and third period T3.

In step S11, for example, in first period T1 and third period T3 of measurement period Tm, three-way electromagnetic valve 22 is in the second state and exposer 20 exposes the plurality of sensors 10 to the reference gas. On the other hand, in second period T2 of measurement period Tm, three-way electromagnetic valve 22 is in the first state and exposer 20 exposes sensors 10 to the sample gas. Accordingly, the plurality of sensors 10 are exposed to the reference gas only in first period T1 and third period T3 of measurement period Tm, and to the sample gas only in second period T2 of measurement period Tm. As a result, the detection signal obtained in step S12 changes, for example, as shown in FIG. 5.

In the example shown in FIG. 5, first, in first period T1 when sensor 10 is exposed to the reference gas, the value of the detection signal hardly changes. Next, in second period T2 when sensor 10 is exposed to the sample gas, sensing section 11 of sensor 10 adsorbs the odorant contained in the sample gas, whereby the value of the detection signal changes (e.g., rises). In third period T3 when sensor 10 is exposed to the reference gas again, the adsorbed molecules are desorbed from sensing section 11 of sensor 10, whereby the value of the detection signal having changed in the second period attempts to return to the reference value. For example, the reference value corresponds to the value of the detection signal before the start of changing due to the exposure of sensor 10 to the sample gas (i.e., the value immediately before the start of second period T2 or at the end of first period T1). In this manner, a pulsed detection signal is obtained by the one-time-exposure of the plurality of sensors 10 to the sample gas. While being pulsed in a convex shape in the example shown in FIG. 5, the detection signal may be pulsed in a concave shape.

The lengths of first period T1, second period T2, and third period T3 are not particularly limited, and set, for example, in accordance with the types of the plurality of sensors 10 and the kind of the molecules to be identified.

Next, extractor 50 extracts features from the plurality of detection signals obtained by obtainer 40 in step S12 (step S13). One, two, or more features may be extracted from each detection signal. That is, one or more features may be extracted from each detection signal. The one or more features extracted from each detection signal include, for example, at least one of the amount of change, the rate of change, or the slope of the detection signal. Being susceptible to the interaction between the odorant and sensor 10, such a feature can increase the accuracy in the determination and identification, which will be described later. Note that the type of the feature described above is an example, and the types of one or more features extracted from the detection signals are not particularly limited. For example, one or more features may include signal values of the detection signals at a certain time point.

In the example shown in FIG. 5, amount ΔV of change of each detection signal corresponds to the difference (VH−VL) between the minimum value VL and the maximum value VH of the detection signal. The rate of change of the detection signal corresponds to the ratio (ΔV/VL) of amount ΔV of change to the minimum value VL. Slope SU of the detection signal represents the amount of change in the value of the detection signal per unit time between two predetermined time points. While being slope SU of the detection signal in second period T2 in the example shown in FIG. 5, the slope of the detection signal may be the slope of the detection signal in third period T3. The rate of change of each detection signal may be the ratio (ΔV/VH) of amount ΔV of change to the maximum value VH.

Even when being exposed to the same gas, the plurality of sensors 10 with different sensitivity characteristics output different detection signals, from which different features can be extracted.

Next, determiner 60 determines, for each of the plurality of sensors 10, whether this sensor 10 is anomalous based on a feature extracted from the detection signal output from this sensor 10 (step S14). Specifically, determiner 60 determines, for each of the plurality of sensors 10, whether this sensor 10 is anomalous based on whether a feature associated with this sensor 10 extracted by extractor 50 in step S13 meets a predetermined requirement. That is, determiner 60 determines whether each of the plurality of sensors 10 is an anomalous sensor whose feature extracted from its detection signal fails to meet the predetermined requirement. In step S13, if two or more features are extracted from one detection signal, for example, any one of the two or more features of the one detection signal is used for the determination. The one feature is, for example, the amount of change, rate of change, or slope of the detection signal.

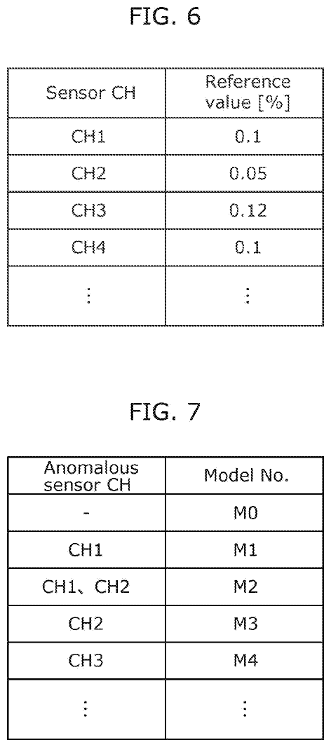

FIG. 6 shows example determination data to be used for determination by determiner 60. In the example shown in FIG. 6, determination data contains the identification information (e.g., the channel (CH) number) on sensor 10 and a reference value as an example of the predetermined requirement for the determination in association with each other. For example, determiner 60 refers to determination data stored in storage 90, and determines sensor 10, whose associated feature is lower than or equal to the reference value, to be anomalous. While the lower limit of the reference value is defined as the predetermined requirement in the example shown in FIG. 6, the upper limit of the reference value may be defined as the predetermined requirement or both the upper and lower limits (i.e., the reference range) of the reference value may be defined.

In the example shown in FIG. 6, the reference value associated with sensor 10 at CH1 as an example of the “first sensor” is different from the reference value associated with sensor 10 at CH2 as an example of the “second sensor”. In this manner, the respective reference values associated with the plurality of sensors 10 may be different from each other. Accordingly, whether each sensor is anomalous can be determined under the determination criteria associated with the sensitivity characteristics of sensor 10, which increases the determination accuracy. Note that all the respective reference values associated with the plurality of sensors 10 may be the same.

After that, selector 70 selects one of the plurality of trained logical models for identifying an odorant, which are different from each other, based on a result of determination by determiner 60 in step S14 (step S15). Selector 70 selects one of the plurality of trained logical models stored in advance in storage 90.

FIG. 7 shows example selection data used for selection by selector 70. In the example shown in FIG. 7, the determination data contains the following identification information in association with each other. The first is the identification information (e.g., the channel (CH) number) on sensor 10 determined to be anomalous by determiner 60, and the second is the identification information (e.g., the model number) on the selected trained logical model. For example, determiner 60 refers to the selection data, and selects the trained logical model associated with sensor 10 determined to be anomalous. When the plurality of CHs like “CH1 and CH2” in FIG. 7 are raised as “anomaly sensor CH” and all the raised CHs are anomalous, selector 70 selects the trained logical model associated with the plurality of CHs. Note that the selection data may contain the information on sensor 10 determined not to be anomalous (i.e., to be normal), instead of sensor 10 determined to be anomalous, in association with a trained logical model.

The trained logical model associated with sensor 10 determined to be anomalous by determiner 60 contains, for example, no input associated with this sensor 10. The trained logical model associated with sensor 10 determined to be anomalous can be built, for example, by machine learning on the logical model containing no input associated with this sensor 10.

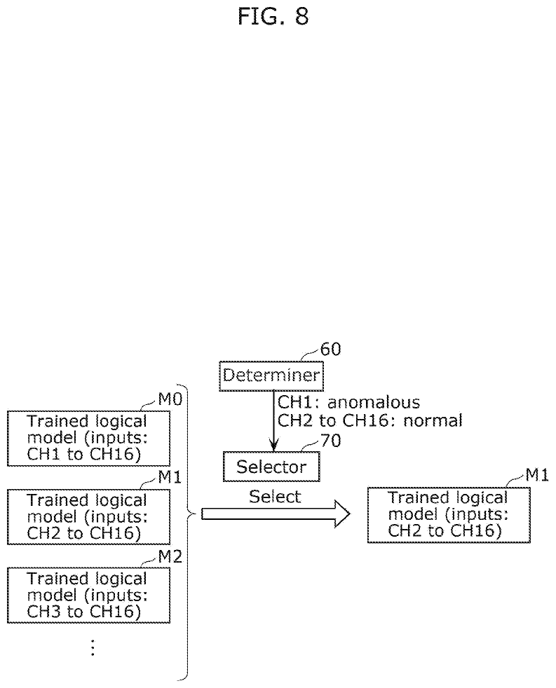

FIG. 8 is for illustrating an outline of the selection processing by selector 70. In the example shown in FIG. 8, sixteen sensors 10 at CHs of CH1 to CH16, out of which sensor 10 at CH1 malfunctions and has a little change in its detection signal even when being exposed to the sample gas.

Selector 70 receives, from determiner 60, a result of determination indicating that sensor 10 at CH1 is anomalous and sensors 10 at CH2 to CH16 are not anomalous. For example, selector 70 refers to the selection data shown in FIG. 7 and selects trained logical model M1 associated with sensor 10 at CH1 determined to be anomalous, out of the plurality of trained logical models M0, M1, M2, and . . . . In the example shown in FIG. 8, trained logical model M1 contains, as inputs, no feature extracted from the signal output from sensor 10 at CH1 determined to be anomalous, but the respective features extracted from the signals output from sensors 10 at CH2 to CH16 determined not to be anomalous. That is, in this case, selector 70 selects trained logical model M1 containing no input associated with sensor 10 at CH1 determined to be anomalous in step S14.

Note that selector 70 may select a trained logical model containing an input associated with sensor 10 determined to be anomalous in step S14. For example, assume that trained logical model M0 contains inputs associated with all the plurality of sensors 10. Selector 70 may select trained logical model M0 for sensors 10 associated with inputs weighted with a predetermined threshold or less, even if this sensor 10 is determined to be anomalous. In this case, even when the odorant identification is performed using trained logical model M0, the input associated with anomalous sensor 10 is less weighted, which hardly degrades the identification accuracy. This can reduce the number of the trained logical models to be stored in advance in storage 90.

For example, selector 70 may select a trained logical model containing an input associated with sensor 10 determined to be anomalous in step S14 and weighted with a predetermined threshold or less. The trained logical model, which contains the input associated with sensor 10 determined to be anomalous and is weighted with the predetermined threshold or less, is generated, for example, based on trained logical model M0 containing inputs associated with all the plurality of sensors 10. First, trained logical model M0 is built, for example, by machine learning on the logical model containing inputs associated with all the plurality of sensors 10. Next, modification is made to coefficients, for example, corresponding to inputs associated with one or more sensors 10 in trained logical model M0 to generate a trained logical model which receives inputs associated with one or more sensors 10 and weighted with a predetermined threshold or less. In this case, there is no need to perform machine learning for each trained logical model to generate the plurality of trained logical models. Accordingly, the plurality of trained logical models can be prepared easily.

Next, identifier 80 identifies the odorant contained in the sample gas, based on the plurality of detection signals obtained in step S12 and the trained logical model selected in step S15 (step S16). Identifier 80 receives, as inputs, features extracted from the plurality of detection signals in step S13, using the trained logical model selected in step S15, and outputs a result of identifying the odorant contained in the sample gas.

As described above, odor identification system 100 selects one trained logical model to be used for identifying the odorant contained in the sample gas, based on a result of determination as to whether each of the plurality of sensors 10 is anomalous. The trained logical model used for the odorant identification may be different between the following two cases. In the first case, all the plurality of sensors 10 are normal. In the second case, the plurality of sensors 10 include sensor 10 that outputs an anomalous signal due to a malfunction, a deterioration, a damage, or other problems. Even when the plurality of sensors 10 include anomalous sensor 10, the accuracy in identifying the odorant less deteriorates by selecting the trained logical model associated with sensor 10 determined to be anomalous. For example, even if any of a plurality of sensors 10 becomes anomalous like malfunctioning, deteriorating, or damaged due to long-term use, the accuracy in identifying the odorant less deteriorates. For example, even if any of the plurality of sensors 10 outputs an anomalous signal (e.g., a signal with an excessively low or high value) indicating an odorant contained in the sample gas, the accuracy in identifying the odorant less deteriorates.

### [Other Example Operations]

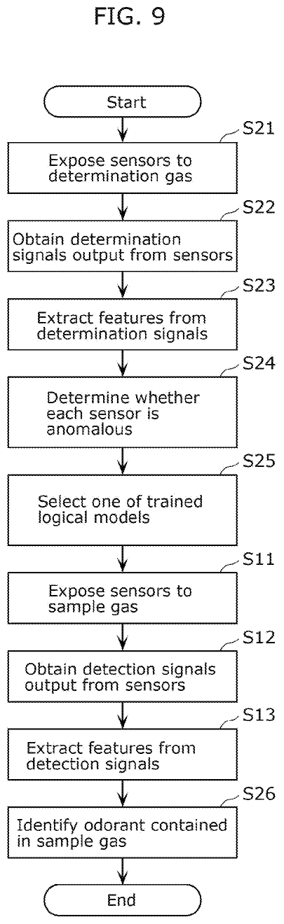

In the example operation described above with reference to FIG. 4, determiner 60 uses features extracted from the plurality of detection signals to determine whether the plurality of sensors 10 are anomalous. The operation is however not limited thereto. FIG. 9 is a flowchart for illustrating another example operation of odor identification system 100 according to this embodiment. In the other example operation shown in FIG. 9, determiner 60 uses features extracted from a plurality of determination signals different from the plurality of detection signals to determine whether the plurality of sensors 10 are anomalous. In the following description of the other example operation, differences from the example operation described above will be mainly described, and the description of the common matters will be omitted or simplified.

In the following description, step S22 is an example of the “obtaining a plurality of determination signals”. Step S24 is an example of “determining”. Step S25 is an example of “selecting”. Step S26 is an example of “identifying”.

As shown in FIG. 9, first, exposer 20 exposes the plurality of sensors 10 to a determination gas with a predetermined humidity based on the control by controller 30 (step S21). Exposer 20 exposes the plurality of sensors 10 to the determination gas through the same operation as in step S11, using the determination gas instead of the sample gas used in step S11 described above. The predetermined humidity is 40% or more, for example, and may range from 40% to 60%. The determination gas is prepared, for example, by controlling the humidity of an inert gas, such as nitrogen. If exposer 20 also functions to control the humidity of the gas to be introduced into housing 21, the humidity of the gas to be introduced into housing 21 may be controlled to serve as the determination gas. For example, in the periods before and after the period of exposing the sensors to the determination gas, exposer 20 may expose the plurality of sensors 10 to a gas, such as the reference gas, with a predetermined humidity (e.g., 0%) or less.

Next, obtainer 40 obtains a plurality of determination signals output from a plurality of sensors 10 exposed to the determination gas in step S21 (step S22).

The plurality of sensors 10 using sensing sections 11 are highly responsive to the water molecules, and thus have outputs changeable in accordance with the humidity. Like the time change in the intensity of the detection signal shown in FIG. 5, the intensity of the signal to be output is changed by exposing associated sensor 10 to the determination gas.

Next, extractor 50 extracts features of the plurality of determination signals obtained by obtainer 40 in step S22 (step S23). Extractor 50 extracts one feature from the determination signal associated with one sensor 10. Extractor 50 extracts, as a feature, the amount of change, the rate of change, or the slope of the determination signal, for example. Extractor 50 may extract, as a feature, a signal value at a certain time of the determination signal.

Next, determiner 60 determines, for each of the plurality of sensors 10, whether this sensor 10 is anomalous based on a feature extracted from a determination signal output from this sensor 10 (step S24). Next, selector 70 selects one of the plurality of trained logical models for identifying an odorant, which are different from each other, based on a result of determination by determiner 60 in step S24 (step S25). The determination in step S24 and the selection in step S25 are performed in the same manner as in step S14 and step S15, respectively.

Next, the same operations as in step S11 to step S13 described above are performed. Identifier 80 then identifies the odorant contained in the sample gas, based on the plurality of detection signals obtained in step S12 and the trained logical model selected in step S25 (step S26). Identifier 80 receives, as inputs, features extracted from the plurality of detection signals in step S13 using the trained logical model selected in step S25, and outputs a result of identifying the odorant contained in the sample gas. Note that each of the operations of step S11 to step S13 may be performed at any time before step S26.

As described above, in this example operation, the features extracted from the determination signals output from the plurality of sensors 10 exposed to the determination gas with the predetermined humidity are used as the feature for determining whether the plurality of sensors 10 are anomalous. With the use of highly responsive water molecules of each sensor, the anomaly of this sensor 10 can be determined with high accuracy. In addition, since the determination gas with a certain humidity is used, the determination signal is less influenced by a disturbance, such as the environment, which stabilizes a result of determination as to whether sensor 10 is anomalous.

### EXAMPLES

Next, the present disclosure will be described specifically based on examples. The present disclosure is however not limited to the following examples. The following shows a result of a test of identifying the odorant contained in the sample gas.

### [Obtainment of Detection Signals and Building of Trained Logical Model]

Sixteen sensors 10 at CH1 to CH16 in housing 21 were used. First, the respective detection signals output from sixteen sensors 10 are obtained, and a plurality of trained logical models were built using the obtained detection signals.

### [Sensor]

Sensors 10 including sensing sections 11 made of different materials (specifically, resin materials) were used as the sixteen sensors. In addition, sensing sections 11 are made of materials each containing a resin material and conductive particles dispersed in the resin material.

### [Gas]

Nitrogen with a humidity of 0% was used as the reference gas. The following three-types of sample gases A to C were used as the sample gas. Each of the sample gas contains any of three types of odorants that are reference odors for odor determination at an odor intensity level of 2 in nitrogen. Specifically, sample gases A to C contain the following odorants.
Sample gas A: β-phenylethyl alcohol (at a concentration of 1.659 ppm)
Sample gas B: methyl cyclopentenolone (at a concentration of 2.033 ppm)
Sample gas C: isovaleric acid (at a concentration of 4.749 ppm)

### [Obtainment of Detection Signals for Training]

By the method described above in step S11, sixteen sensors 10 were subjected to the following operation 66 times. In the operation, sixteen sensors 10 were exposed to sample gases A to C and the respective detection signals (i.e., the detection signals for one set) output from sixteen sensors 10 were obtained. For each of sample gases A to C, 66 sets of detection signals for training were obtained.

### [Obtainment of Detection Signals for Identification Test]

By the method described above in step S11, sixteen sensors 10 were subjected to the following operation 44 times under the following five conditions. In the operation, sixteen sensors 10 were exposed to sample gases A to C and the respective detection signals output from sixteen sensors 10 were obtained. For each of sample gases A to C, 44 sets of detection signals were obtained under each condition.
Condition 1: all sensors 10 are normal
Condition 2: part of wires is disconnected in sensor 10 at CH8
Condition 3: part of wires is disconnected in sensor 10 at CH12
Condition 4: part of wires is disconnected in sensor 10 at CH16
Condition 5: part of wires is disconnected in sensors 10 at CH8, CH12, and CH16

### [Extraction of Features]

The rate of change, the amount of change, and the slope of each of the obtained detection signals were extracted as features.

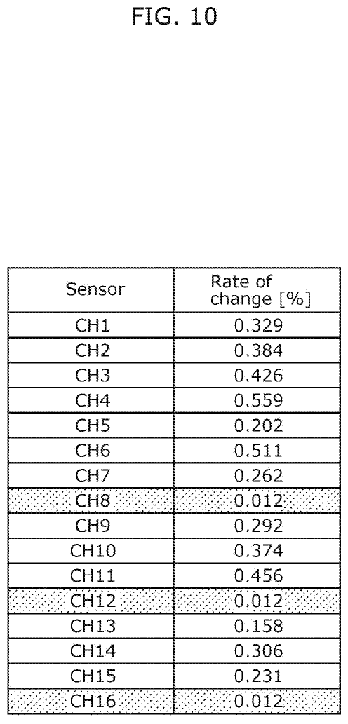

The following was found as a result of checking the rates of change in the features extracted from the detection signals for the identification test. The rate of change of the detection signal of sensor 10 was 0.1% or less in sensor 10 at CH8 in which part of wires was disconnected. On the other hand, the rates of change of the respective detection signals of normal sensors 10 were higher than 0.1%. FIG. 10 shows an example rate of change of each detection signal. FIG. 10 shows rates of change of the plurality of detection signals for the identification test obtained under condition 5. As shown in FIG. 10, the rates of change of the detection signals were 0.1% or less in sensors 10 at CH8, CH12, and CH16 in which part of wires was disconnected (see the dotted sections in FIG. 10). On the other hand, the rates of change of the detection signals were 0.1% in sensors 10 at CHs other than CH8, CH12, and CH16. Accordingly, for example, the reference values for determining whether all sensors 10 are anomalous are set to 0.1%. Sensors 10 at CH8, CH12, and CH16 can be then determined to be anomalous, in which the rates of change are lower than or equal to the reference value and part of wires is disconnected.

### [Building of Trained Logical Models]

Five types of trained logical models containing the following different inputs are each built by performing machine learning on a logical model, using the odorant contained in the sample gas and the features extracted from the detection signal for training associated with the odorant, as training data. The random forest was used as the logical model.
Trained logical model 1: built by machine learning on a logical model containing inputs associated with all sensors 10
Trained logical model 2: built by machine learning on a logical model containing inputs associated with sensors 10 at CHs other than CH8
Trained logical model 3: built by machine learning on a logical model containing inputs associated with sensors 10 at CHs other than CH12
Trained logical model 4: built by machine learning on a logical model containing inputs associated with sensors 10 at CHs other than CH16
Trained logical model 5: built by machine learning on a logical model containing inputs associated with sensors 10 at CHs other than CH8, CH12, and CH16

In trained logical model 1, the inputs associated with sensors 10 at CH8, CH12, and CH16 were weighted more than inputs associated with sensors 10 at CHs other than CH8, CH12, and CH16.

### [Identification Test]

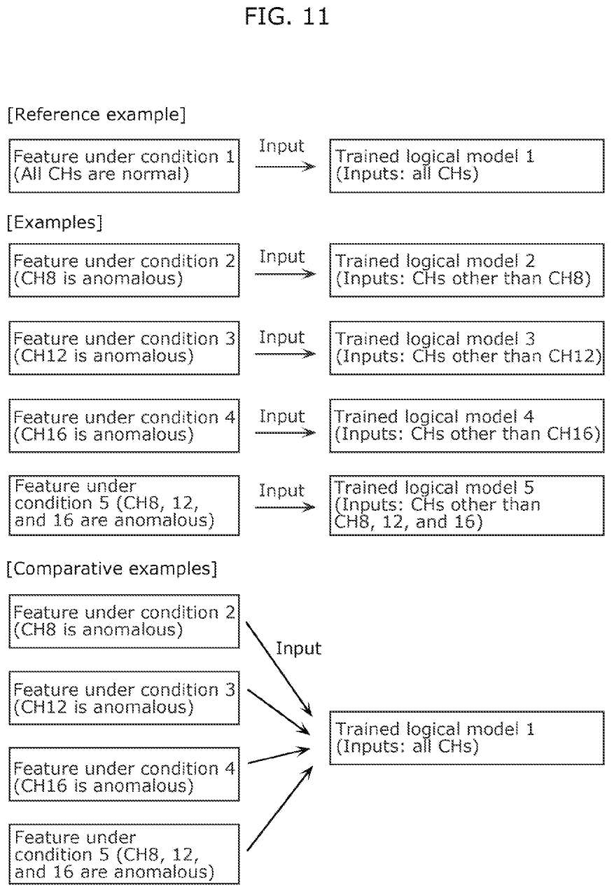

A test of identifying the odorant contained in the sample gas was conducted by inputting the features extracted from the detection signals for the identification test into the trained logical model built as described above. FIG. 11 is for illustrating how to conduct the odorant identification test.

As shown in FIG. 11, in the reference example, the features extracted from the detection signal obtained under condition 1 was input to trained logical model 1.

In the examples, the features extracted from the detection signals obtained under conditions 2 to 5 were input to trained logical models 2 to 5, respectively. That is, in the examples, the odorant identification was performed using the trained logical models containing no inputs associated with sensors 10 determined to be anomalous. Accordingly, in the examples, the odorant identification was performed without using, as inputs, the features associated with sensors 10 determined to be anomalous as well.

In the comparative examples, the features extracted from the detection signals obtained under conditions 2 to 5 were input to trained logical model 1. That is, in the comparative examples, the odorant identification was performed using the trained logical models containing inputs associated with sensors 10 determined to be anomalous.

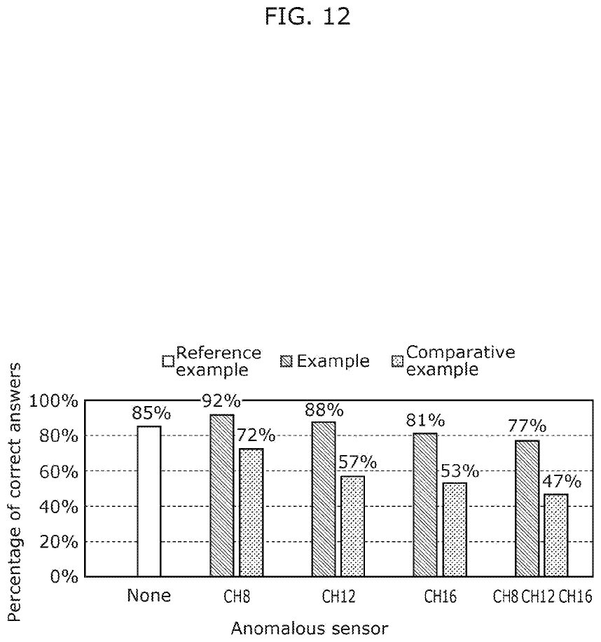

FIG. 12 shows a result of the identification test. FIG. 12 shows a result of identifying the odorant in the identification test. In FIG. 12, the percentages of correct answers are of the output odorants obtained by inputting the features associated to 44 sets of sample gases A to C to the trained logical model. In FIG. 12, the white bar represents the reference example, the slashed bars represent the examples, and dotted bars represent the comparative examples.

As shown in FIG. 12, the percentages of correct answers are lower in the comparative example than in the reference example. The comparative examples uses same trained logical model 1 as in the case were all sensors 10 are normal, although some of sensors 10 are anomalous. This may be because the features associated with anomalous sensors 10 are also input to the trained logical model.

By contrast, if some of sensors 10 are anomalous, the percentages of correct answers are higher in the examples than in the reference example, that is, the percentages of correct answers equivalent to those in the reference example are kept. If some of sensors 10 are anomalous, the examples use trained logical models 2 to 5 containing no inputs associated with sensors 10 determined to be anomalous. That is, it was confirmed in the example that the deterioration in the accuracy in identifying the odorant was less reduced, even when anomalous sensors 10 were included.

### [Other Variations]

While the odor identification system and the odor identification method according to one or more aspects have been described above based on the embodiments, the present disclosure is not limited to the embodiment and the examples. The present disclosure may include forms obtained by various modifications to the foregoing embodiment and examples that can be conceived by those skilled in the art or forms achieved by freely combining some of the elements in the foregoing embodiment and examples without departing from the scope and spirit of the present disclosure.

In the embodiment and examples described above, obtainer 40 obtains the plurality of detection signals output from the plurality of sensors 10 directly. The configuration is however not limited thereto. For example, obtainer 40 may obtain the plurality of detection signals and the plurality of determination signals output from the plurality of sensors 10 in step S12 and step S22 via a network. In this case, for example, each of detection device 101 and identification device 102 includes a communication circuit and communicate with each other via the network. The communications may be established in a wireless or wired manner. The communication method (i.e., the communication protocol) is not particularly limited. The communications may be established through a wide-area communication network, such as the Internet.

In the embodiment described above, the processing executed by a certain processor may be executed by another processor. The plurality of processing may be performed in a different order or in parallel. The assignment of the elements of odor identification system 100 into the plurality of devices is a mere example. The elements of a device (e.g., one of detection device 101 and identification device 102) may be included in another device (e.g., the other of detection device 101 and identification device 102). For example, the elements of one of detection device 101 and identification device 102 may be included in the other of detection device 101 and identification device 102. For example, odor identification system 100 may include one device, or three or more devices.

For example, the processing described above in the embodiment may be concentrated using a single device (or system) or distributed using a plurality of devices. The program described above may be executed by a single processor or a plurality of processors. In short, concentrated or distributed processing may be performed.

For example, in the embodiment described above, some or all of the elements of the odor identification system according to the present disclosure may be achieved by dedicated hardware or by executing the software programs suitable for the elements. The elements may be achieved by a program executor, such as a CPU or a processor, reading out software programs stored in a recording medium, such as an HDD or a semiconductor memory, and executing the read-out programs.

The elements of the odor identification system according to the present disclosure may be one or more electronic circuits. Each of the one or more electronic circuits may be a general-purpose circuit or a dedicated circuit.

Examples of the one or more electronic circuits may include a semiconductor device, an integrated circuit (IC) or a large-scale integration (LSI) circuit. The IC or LSI circuit may be integrated on one chip or a plurality of chips. While the IC or LSI circuit is named here, the integrated circuit may be named differently depending on the degree of integration, and may be referred to a system LSI circuit, a very-large-scale integration (VLSI) circuit, or an ultra-large-scale integration (ULSI) circuit. A field programmable gate array (FPGA) programmable after the manufacture of an LSI circuit may be employed for the same purpose.

Note that these general and specific aspects of the present disclosure may be implemented using a system, a device, a method, an integrated circuit, or a computer program. Alternatively, the aspects may be implemented using a non-transitory computer-readable recording medium, such as an optical disk, HDD, or a semiconductor memory, storing the computer program. The aspects may be implemented using any combination of systems, devices, methods, integrated circuits, computer programs, or recording media.

For example, the present disclosure may be directed to an odor identification method to be executed by a computer included in the odor identification system, for example, or to a program for causing a computer to execute such the odor identification method. The present disclosure may also be directed to a non-transitory computer-readable recording medium having such the program recorded thereon.

### INDUSTRIAL APPLICABILITY

The odor identification system and the odor identification method according to the present disclosure are useful for a system for identifying an odorant contained in a sample gas, and can be used for identifying various odors of foods, human body, or building, for example.

1. An odor identification method of identifying an odorant contained in a sample gas, using a plurality of sensors each outputting a signal according to an adsorption concentration of molecules, the odor identification method comprising:
obtaining a plurality of detection signals output from the plurality of sensors exposed to the sample gas;
determining, for each of the plurality of sensors, whether the sensor is anomalous based on a feature extracted from the signal output from the sensor;
selecting one of a plurality of trained logical models that are for identifying the odorant and different from each other, based on a result of determination in the determining; and
identifying the odorant contained in the sample gas, based on the plurality of detection signals obtained in the obtaining of the plurality of detection signals and the one of the plurality of trained logical models selected in the selecting.

2. The odor identification method according to claim 1, wherein
n the determining, a feature extracted from each of the plurality of detection signals is used as the feature.

3. The odor identification method according to claim 1, further comprising:
obtaining a plurality of determination signals output from the plurality of sensors exposed to a determination gas with a predetermined humidity, wherein
in the determining, a feature extracted from each of the plurality of determination signals is used as the feature.

4. The odor identification method according to claim 1, wherein
the determining includes determining, for each of the plurality of sensors, whether the sensor is anomalous, based on whether the feature associated with the sensor meets a predetermined requirement, and
a predetermined requirement associated with a first sensor of the plurality of sensors is different from a predetermined requirement associated with a second sensor of the plurality of sensors.

5. The odor identification method according to claim 1, wherein
the selecting includes selecting, as the one of the plurality of trained logical models, a trained logical model containing no input associated with a sensor determined to be anomalous in the determining.

6. The odor identification method according to claim 1, wherein
the selecting includes selecting, as the one of the plurality of trained logical models, a trained logical model in which an input associated with a sensor determined to be anomalous in the determining is weighted with a predetermined threshold or less.

7. The odor identification method according to claim 1, wherein
in the determining, the feature includes an amount of change, a rate of change, or a slope of the signal output from the sensor.

8. The odor identification method according to claim 1, wherein
the obtaining of the plurality of detection signals includes obtaining a plurality of detection signals output from the plurality of sensors via a network.

9. An odor identification system for identifying an odorant contained in a sample gas, using a plurality of sensors each outputting a signal according to an adsorption concentration of molecules, the odor identification system comprising:
an obtainer that obtains a plurality of detection signals output from the plurality of sensors exposed to the sample gas;
a determiner that determines, for each of the plurality of sensors, whether the sensor is anomalous based on a feature extracted from the signal output from the sensor;
a selector that selects one of a plurality of trained logical models that are for identifying the odorant and different from each other, based on a result of determination by the determiner; and
an identifier that identifies the odorant contained in the sample gas, based on the plurality of detection signals obtained by the obtainer, and the one of the plurality of trained logical models selected by the selector.
